(19) United States
(12) Patent Application Publication
(10) Pub. No.: US 2003/0060603 A1
(43) Pub. Date: Mar. 27, 2003

(54) Antibodies generated against polypeptide targets expressed from polynucleotide administration

(75) Inventor: Moncef Jendoubi, San Francisco, CA (US)

Correspondence Address: ORRICK, HERRINGTON & SUTCLIFFE, LLP, 4 PARK PLAZA, SUITE 1600, IRVINE, CA, 92614-2558, US

(73) Assignee: Milagen, Inc.

(21) Appl. No.: 10/232,777

(22) Filed: Aug. 30, 2002

Related U.S. Application Data
(60) Provisional application No. 60/316,708, filed on Aug. 31, 2001.

(51) Int. Cl. C07K 1/00; C07K 14/00; C07K 17/00

(52) U.S. Cl. 530/350

(57) ABSTRACT

The present invention is directed to the production of antibodies drawn to the gene products of polynucleotides expressed through genetic immunization. The antibodies are correlated on a one-to-one basis with the gene sequences encoding the gene products to which the antibodies are specific. The polynucleotides are placed in recombinant vector constructs for expression and may be used without knowledge of the open reading frame. The antibodies may be used to analyze gene expression and protein expression profiling for research, diagnostics, or therapeutics.

### RELATED INFORMATION

This application claims priority of Provisional Application Serial No.: 60/316,708 filed on Aug. 31, 2001. The priority of the prior application is expressly claimed, and the disclosure of the prior application is hereby incorporated by reference in their entirety.

### FIELD OF INVENTION

The present invention relates to a method of producing antibodies produced against any amino acid sequence (any succession and number of amino acids) derived from the expression of any polynucleotide sequence (any succession of a triplet of nucleotides). Polyclonal antibodies are made in vivo in animals capable of an immune response. The antibodies are used in vitro or in vivo to gain knowledge and to determine utility of the generated antibody, its recognized target, and the expressed polynucleotide sequence. The polynucleotides are from several sources, including pathogens, mammals, animals and plants, and the antibodies generated through this process can be used for research and diagnostic and therapeutic applications.

### BACKGROUND OF THE INVENTION

Over the past fifteen years, billions of dollars have been spent in manpower, technology innovation, bioinformatics tools, gene sequences, cDNA libraries, genomic sequencing and worldwide databases of expressed sequence tags (ESTs). The sequencing of the human genome will eventually facilitate the discovery of genes involved in a variety of diseases, and holds great potential for drug discovery.

The isolation and sequencing of a tremendous number of gene sequences does not address the complexity of the protein, protein evolution or expression patterns, or how proteins interact to accomplish a physiological function or initiate a pathological process. The sequencing of the human genome does not answer questions of how many transcripts and proteins are derived from each gene, what is the function of each protein, and ultimately how proteins interact with each other in normal and pathological conditions.

The life of a cell (i.e. growth, division, death, migration, shape, nutrition, metabolism, stress response, defense, etc.) is a dynamic process in which the cell constantly reacts to its environment. Each cell makes a pool of specific proteins. Protein pools vary in composition and in amount depending on the cell and tissue type. Proteins are often associated to carry out their function. The intrusion of a physical, chemical or pathological agent in our body immediately affects the subtle molecular balance within the cell, and eventually results in a disease state. The response to an outside stimulus may turn on, off, or regulate gene expression, and may change in the amount of a transcript, the amount, form, or composition of a gene product, and may cause modification of the resulting protein.

Recently, the study of proteins, referred to as “proteomics,” aimed to study the total protein complement of a genome. Proteomic approaches typically involve 2-D gel electrophoresis coupled to mass spectrometry (MS) techniques. The general principle of 2-D PAGE coupled to MS is that a protein complex can be separated by 2-D gel, and MS applied to peptides derived from the trypsin digestion of individual protein spots. Once the peptide sequence is identified, the corresponding nucleotide sequence is derived and compared to genomic databases. These approaches require innovative computational tools and methods to process, interpret, and analyze the tremendous amount of data generated from the analysis of any significant number of proteins.

However, genomics and proteomics approaches have drawbacks when applied to drug target discovery. While available sequence information, databases, DNA chip data points and comparative analysis of gene sequences between various species are available, the information on the biological function of each protein is not readily obtained. Similarly, while proteomic studies are useful in some applications, proteomics is limited in the discovery of gene sequences and correlate to protein function. Thus, there is a great need to technologies that would greatly facilitate the correlation between the gene sequence, the gene product, and the function.

The most favorable approach would be to generate antibodies in mammals to expressed gene products. These antibodies will then be used to as tools for research, diagnostic, and therapeutic applications.

Most drug targets in the market were initially discovered and validated using antibodies as a basic discovery tool. Typically, an antibody is produced to take advantage of the capability to specifically bind an antigen, usually a protein, that is known to be involved in a biological process. The practical advantage of an immunological strategy to the study of biological processes is that the immune system is capable of producing binding proteins called “antibodies” that are produced when the immune system encounters an “immunogen” or “antigen.” The antibodies are highly reactive and specific to the antigen and are unique and powerful reagents for a wide variety of applications. As with most biological reagents, the preparation and use of antibodies requires an efficient production methodology and an application that is well suited to the analytical rationale to which the reagent will be applied. Because of the unique nature of the antigen-antibody reaction, antibodies have been used in a large number of different analytical or diagnostic methodologies to achieve a variety of analytical or diagnostic goals. The antibody provides the researcher with the ability to selectively bind the antigen and to qualitatively or quantitatively detect the presence of the antigen in a sample and used to physically separate the antigen from a mixture of compounds contained in a sample. Thus, when a particular interest in an antigen exists, an antibody specific to the antigen is an invaluable tool for studying the antigen because of the binding specificity. For example, (1) a gene product differentially is identified that is in a cell type and which seems to play an important physiological role; (2) the suspected gene product is purified and an animal is immunized to make specific antibodies. Subsequently, the antibodies are with the gene product used (a) to characterize the desired gene product; (b) to study the gene product function; and (c) ultimately, to use the antibodies to screen an expression cDNA library in E. Coli to identify the gene sequence encoding the protein. There are numerous cases where the gene product and its sequence were discovered and validated based on this approach.

Most antibodies are produced by immunizing an animal, usually a non-human vertebrate and frequently a mouse, rabbit, or goat, with an antigen of interest and encouraging the animal's immune system to generate antibodies specific to the antigen that may then be detected in the animal's sera. If the antigen causes a strong enough immune response, usually requiring the passage of time and a series of “booster” immunizations wherein the antigen is repeatedly injected to provide a strong immune response, the antibody can be produced in useful quantities. This process typically requires several weeks and a reasonably large investment in labor and resources. Because of the investment of time and resources required to produce antibodies to selected antigens, antibodies generally are not produced to antigens unless the significance of an antigen is recognized. When the significance of a protein-related antigen is known, the gene sequence that encodes the protein is usually also determined. Thus, in many traditional analyses, the protein is characterized, an antibody obtained, and the gene sequence expressing the protein is determined only when the protein is suspected of being biologically significant.

As noted above, different strategies for the use of antibody-based immunoassays has led to a wide variety of applications wherein the antibody is used as a tool to characterize the protein target of interest. For example, an enzyme-linked immunoabsorbent assay (ELISA) is an assay technique that is particularly useful to diagnose antigens such as viruses, bacteria, or proteins, including antibodies, in sera or in other biological samples. Particularly where quantitative analysis is desired, the antigen or antibody is absorbed onto a solid surface, such as the well of a microtiter plate, so that the reaction of a particular antibody-antigen pair can be analyzed. Using any of several variations on the ELISA, a quantitative or qualitative measurement of a particular antibody-antigen interaction can be made.

In most applications of the ELISA technique, the antigen is a well-characterized protein or polypeptide fragment whose biological significance is recognized. Where the specific nature and function of the protein is known, the gene sequence that encodes the protein may or may not be known. If the nature of the antigen is not known, while the information derived from the use of the antibody are relevant, then several techniques known to skilled individuals can be readily used to identify the gene sequence which encodes the gene product. The antibodies are well known as precious tools invaluable for discovery and validation. Indeed, the present invention relates to a method to generate antibodies to known and unknown polypeptide at a large scale, and to their use for discovery and validation of pharmaceutical disease targets.

Recently, the prospect of sequencing the entire genome of an organism has led to a great hope for discovering new genes whose expression affects a variety of diseases or physiological conditions. However, given the large number of available genes, the absence of information regarding proteins encoded by these genes, and the absence of antibodies as tools to characterize the gene products and their function, the analysis of gene function in actual physiological events remains a very slow process. There is a great need to generate antibodies to allow the accomplishment of a variety of functional studies to define the physiological role of proteins and all manifestations of gene products expression.

Unfortunately, as noted above, the production of antibodies to gene products has traditionally been an arduous and time consuming task requiring injections of a protein-antigen into a vertebrate animal with a functioning humoral immune system. The choice to endure the expense of time and resources inherent to the production of antibody could be justified because valuable information could be gained from the use of the antibody, assuming a reasonably high degree of confidence that the protein-antigen was biologically significant.

The discovery that the introduction of plasmids containing a gene sequence in the correct open reading frame could stimulate the production of antibodies to the expected expression product, under appropriate circumstances, is an alternate approach to the classical mehtod of production of antibodies. As described in M. A. Barry et al., 1995, “Protection against mycoplasma infection using expression-library immunization,” Nature 377:632-635, plasmids containing a human growth hormone gene under the control of the cytomegalovirus promoter can yield moderate to the antibodies following biolistic injection into the dermal tissue of mice utilizing gold particles as a carrier. However, the process of producing DNA coated microprojectiles is cumbersome and time consuming. Plasmid DNA must be added to a microcentrifuge tube containing gold beads suspended in spermidine, and then precipitated onto the beads by vortexing with calcium chloride. After centrifuging and washing, the DNA coated beads are then mixed with ethanol and sonicated to generate a uniform gold suspension. The suspension is then transferred to a length of Tefzel tubing, coated over the interior surface, and dried. The dried DNA gold particles are then loaded into a helium-powered gene delivery device for biolistic injection. Although faster than the conventional process outlined above, the gold projectile process is still too costly, cumbersome, and labor intensive for use in generating an antibody library of a significant size and diversity.

Alternatively, highly purified plasmids containing a gene, inserted in the correct open reading frame, have been directly injected into the muscle of mice, as described in U.S. Pat. No. 5,589,466. However, this approach only produced a weak initial antibody response, which decreased to almost baseline levels after several weeks. In addition, the repeated cesium chloride centrifugation protocol used to prepare the plasmids for injection is cumbersome and time consuming.

More importantly, DNA-based immunization, is applied to generate antibodies to known proteins where the full coding sequence of the desired gene product is known. Furthermore, the coding nucleotide sequence is always carefully introduced in the correct open reading frame to insure the expression of the correct known protein. Knowing the entire open reading frame, the investigator may choose to sub-clone either the entire desired gene sequence or only a partial fragment of the coding sequence depending on the need. The polynucleotide sequence of the desired gene is placed in the correct open reading frame to ensure that the polynucleotide sequence expresses the right polypeptide. Consequently, when a recombinant vector construct containing a known polynucleotide sequence in the correct open reading frame is delivered to an animal, it expresses the desired polypeptide in the animal. This in turn will generate specific polyclonal antibodies to the expected protein.

The correct cloning of the desired gene or gene fragment in the correct open reading frame requires a substantial experimental effort and manipulation. The time involved to design, construct, verify, optimize and implement one successful recombinant expression vector containing a polynucleotide sequence of a given gene may vary between several weeks and several months. When the recombinant construct is completed 2 to 3 additional months will be needed for successive immunization to generate a satisfactory antibody titer and specificity to the desired gene product. Thus, the production of antibodies based on the DNA immunization is still an intensive labor and time consuming, even when the full sequence and the correct open reading frame are known. Thus, using existing technique antibodies to the expression product of a large number of different polypeptides cannot be obtained in a reasonable period of time and at an affordable cost.

Currently, the total number of genes in the human genome is estimated to be around 40,000 genes and possibly twice as much according to a recent publication (Fred A. Wright et al. Genome Biology, 2:7, 2001). The approximately 40,000 genes contained in the human genome are estimated to generate at least 5-10 folds more transcript isoforms and each transcript isoform is estimated to make several different proteins. Because of the size of this number, the current approach of DNA immunization cannot be used to generate antibodies to a significant portion of the polynucleotide coding sequences—the genome.

A need exists to generate antibodies in high throughput fashion. The need also exists to make this approach, fast, economical, less cumbersome and less time consuming. A need exists for a simple, fast, economical process which produces high titer antibodies against any polypeptide sequence encoded by any given polynucleotide sequence derived from any genome.

There is also a need to produce specific antibody reagents against every expressed polynucleotide sequence in an animal to speed up the characterization of the different gene products made by the human genome to understand their function, their physiological role and to identify pharmaceutical targets for human diagnostics or therapeutics.

Furthermore, there is a need for the ability to correlate the binding of an antigen and an antibody in a rapid, high throughput analysis to the actual polynucleotide sequence that encoded the gene product for which the antibody is specific. There is a need to analyze thoroughly the expression products of the genome of an organism on the basis of their gene-products in different cell types, such as cancer cell lines, primary cell lines, and tissue specific cell lines. A differential screening of the gene products in these cell lines can be useful to identify specific gene-products expressed, up/or down regulated, or absent in these cell lines in normal and pathological conditions.

There is a great need to discover the gene products that are associated with a variety of threatening disorder such as cardiovascular disease, cancers, inflammatory, neurological and infectious diseases to discover the underlying gene functions and their relation with these diseases. Moreover, there is a great need for a method that correlate the gene with its pattern of expression, in different cell types and tissues as well as their cellular localization.

There is a great need to generate a data bank that contains all information related to mammalian gene products, their pattern of expression, gene-products and their isoforms, cellular expression at both the tissular and temporal level, and the gene sequences to which the gene products are correlated.

There is also a need to overcome the recognized impediments to gene discovery, and to possess a readily available biological tag to allow the study of the gene function in vivo. The need also exists for antibodies to accelerate the discovery of the function of novel genes in normal and pathological conditions, to discover and design diagnostic kits to prevent or/and evaluate the progress of a particular pathology, and to discover candidates for therapy against a variety of diseases.

Finally, there is a need for method to yield polynucleotide template of an optimal quality that elicits a strong immune response following polynucleotide sequence immunization.

### SUMMARY OF THE INVENTION

The present invention circumvents the impediments to large-scale antibody generation and enables the production of antibodies to a polypeptide expressed by any polynucleotide sequence in any open reading frame. The present invention takes advantage: (a) of the efficiency and the specificity and the wide immune response against a particular polypeptide of any length and form; including peptides, proteins, protein fragments, carbohydrates, organic molecules, or any immunogen against which the immune system of an animal will produce antibodies; (b) of the use of an animal with an immune system capable of mounting a distinct response against such particular antigens; (c) of the need to analyze gene expression and to discover new genes and new gene expression products, particularly proteins having physiological significance in human disease; (d) the possibility that any polynucleotide sequence from any organism could express at least one polypeptide and possibly more than one polypeptide if the polynucleotide sequence is contained in an appropriate genetic context that would favor the translation of the transcript (s); (e) of the possibility to generate antibodies to possibly any peptide or polypeptide encoded by any polynucleotide sequence at a high throughput scale, (f) of the exquisite nature of specificity of the antibody and their broad use in multiple and diversified assays, including multiplex format for rapid high throughput analysis of biological samples; (g) of the possibility to correlate the antigen present in a the biological sample with the polypeptide expressed by the polynucleotide sequence included into the recombinant construct to the antibodies and the information.

The present invention takes advantage of the fact that any polynucleotide sequence derived from any gene sequence may give rise to several transcripts of different length, with different open reading frames and different starting and stoping codons. This in turn will result in the translation of different polypeptides which could be either isoform polypeptide to each others or distinct polypeptides compared to each other, depending of the used open reading frame of the polynucleotide sequence.

The present invention enables the expression of at least one polypeptide and possibly more than one polypeptide of different size derived from one or more than one open reading frames. The polypeptides of the present invention may have different length and different primary amino acid composition. Thus, the present invention enables the production of antibodies against known and unknown polypeptide sequences expressed from any polynucleotide sequence of any genome of plants, insects, pathogens, or animals.

The present invention also includes the use of antibodies to gain valuable information on the gene product recognized specifically by the antibody made against the polypeptide expressed by the polynuceotide sequence and to correlate the reactivity of the antibody to the original polynucleotide. The present invention has an invaluable advantage to make antibodies to any polypeptide expressed from any polynucleotide sequence.

The antibodies generated by the method of this invention can be used in a variety of screening assays, including the immobilization of the generated antibodies on a solid support, preferably in an array or matrix for high throughput analyses and gene product discovery and validation.

In the method of the invention, antibody arrays are used to examine gene product expression profiling of a given normal or diseased tissue, or sample from a patient with disease or suspected of disease, or for analyzing a cell type before and after exposure to drugs, chemicals, or physical stimuli, such as carcinogens, irradiation, toxic agents, pharmacological agents, and the like. Antibody arrays are also used to discover gene products related any chemical, toxic, or physical agent and/or any other stimulus, to an onset of disease, the progression of disease, the resistance to treatment, or the relationship between toxic agents and abnormally regulated gene products, or virtually any pathological and physiological changes characteristic of a normal or disease state.

The antibodies produced pursuant to this invention allow the examination and analysis of hundreds of gene products all at once, and can be used to analyze a specific physiological or pathological state of a sample, whether derived from the cells, tissue or biological fluid from a host. For example, a human patient may be in a normal or disease state, or exposed to a stimulus or chemical or biological agent, and a sample containing protein(s) secured. Because the sample so secured will contain proteins reflecting the physiological state of the organism, and specifically reflecting the nature of the gene expression underlying a physiological state, analysis of the sample with antibodies produced pursuant to the invention, will demonstrate the specific genes being expressed in a specific physiological state by correlating the binding of antibodies raised from specific polynucleotides to protein in the sample. In this fashion, the methods of the present invention identify and correlate, in a one-to-one relationship, the antibody, the gene product and the polynucleotide sequence (DNA or RNA) coding for the polypeptide that elicited the production of the antibody. The present invention is also related to derivatives of antibody, polypeptide and polynucleotide, agonists, or antagonist and the like recognized by the produced antibodies. The antibodies produced by the present invention can be used in any immunological or biological assay in vivo and in vitro as described in Current Protocols in Immunology by John E. Coligan et al., 1995. Edition of John Wiley and Sons, Inc.

The present invention includes a method of producing antibodies to at least one, and preferably more than one polypeptide, encoded by a polynucleotide sequence delivered to an animal. In a preferred embodiment, the polynucleotide sequence is included in a composition comprises of a recombinant polynucleotide construct encoding the polypeptide, bacterial ribonucleic acids, and may also include bacterial proteins. This composition is delivered to a non-human animal capable of a humoral immune response. Preferably, the non-human animal is a vertebrate and most preferably an out-bred mouse. Although the injection site may vary, the preferred route of injection is inguinal. The composition for immunization may also include additional salt compositions, including EDTA, Tris, SDS, or other suitable salts. The composition may also include bacterial toxins. In the preferred embodiment of the invention, polyclonal murine isotype IgG antibodies are obtained from a single injection wherein the recombinant polynucleotide contains a gene sequence capable of expression in the non-human animal and which may or may not be specifically constructed for expression of a specific polynucleotide from an open reading frame. The recombinant polynucleotide construct may therefore include additional polynucleotide sequences five prime of the polynucleotide expressed upon immunization in the animal. These five prime sequences include, but are not limited to, promoters, start sites, or polynucleotides that are isogenic with the polynucleotide expressed by the construct.

The recombinant polynucleotide construct is delivered to an animal in amounts sufficient to allow the construct to be taken up by the cells of the vertebrate so that sufficient amounts of the encoded protein are produced to induce the production of antibodies to the protein in the vertebrate. Amounts of injected recombinant polynucleotide construct encoding the polypeptide into an animal are preferably in the range of about 1-10 μg, or 2-50 μg, or 10-100 μg, or 500 μg, more preferably in the range of about 200 to about 450 μg, and most preferably in the range of about 250 to about 400 μg. The amount of bacterial RNA injected into the vertebrate is preferably more than 1/10,000 of the amount of polynucleotide sequence encoding the polypeptide, more preferably more than 1/1000 of the amount of polynucleotide sequence encoding the polypeptide, and most preferably more than 1/100 of the amount of the polynucleotide sequence encoding the polypeptide, as determined by weight. It is preferred that the amount of bacterial RNA be less than equal to the amount of polynucleotide sequence encoding the polypeptide, as determined by weight.

Another aspect of the invention also relates to a method for producing polynucleotide templates comprising DNA and/or RNA for genetic immunization of an animal, preferably a rodent. Production of the polynucleotide is achieved by: (1) growing a prokaryotic cells containing a recombinant polynucleotide construct which bears the partial or complete polynucleotide sequence of any given gene; (2) lysing cells containing the recombinant polynucleotide construct to obtain a lysate; (3) treating the lysate to remove insoluble material and obtain the plasmid solution in a non pharmaceutical solution; (4) precipitating the polynucleotide templates containing DNA and RNA to recover the polynucleotide immunizing solution containing the partial or complete polynucleotide sequence of any given gene of interest. The polynucleotide template is produced without organic extractants or/and solvents. The polynucleotide templates containing the recombinant polynucleotide construct is a non-pharmaceutical grade material in a suitable solution for injection and is specifically formulated and designed to stimulate the immune system and allow a strong immune response by the animal against the gene of interest within the recombinant polynucleotide construct.

Another aspect of the invention relates to a method for producing polynucleotide templates containing recombinant polynucleotide construct DNA and RNA suitable for genetic immunization from a microorganism and/or from any transformed cell type. The method is particularly focused on the process for the isolation and purification of hundreds of micrograms of polynucleotide templates containing DNA and RNA sequences from hundreds of different cell clones transformed with recombinant polynucleotide constructs harboring polynucleotide sequence that encodes for a partial or total polypeptide of a given gene. The polynucleotide template containing DNA or RNA is a non-pharmaceutical-grade compound in a solution specially designed as an immunostimulant.

### DETAILED DESCRIPTION OF THE INVENTION

The term “polynucleotide” refers to a succession of a triplet of nucleotides comprising at least 6 triplets and possible of a multiple of 1, 2, 3, 4, 5, 6, 7, 8, 9, 10 triplets or more, including 10, 20, 30, 40, 50, 60, 70, 80, 90, or 100, 200, 300, 400, 500, 600, 700, 800, 900 or 1000 or more and any integral value therein. The total length of the polynucleotide usually varies between 18 nucleotides to several thousand nucleotides.

“Peptide,” “polypeptide,” or “gene product” or “gene expression product” are the broad class of compounds that are produced by transcription and translation of a polynucleotide sequence, regardless of its length, of which a gene is comprised in a cell. These translation products include peptides, proteins, and polypeptides, and may be conjugated with a lipid, a phosphate, a sugar, and the like. The polypeptide may be a full-length protein exhibiting normal folding patterns, or a fragment, truncation, cleavage or other polypeptides form modified by post-translational processing or modification.

The term “antibody” refers to an antibody (e.g., a monoclonal or polyclonal antibody), having specific binding affinity to a peptide, a polypeptide, gene product or a fragment of the gene product of a cell.

The term “antibody fragment” refers to a portion of an antibody, often the hypervariable region and portions of the surrounding heavy and light chains, that displays specific binding affinity for a particular molecule. A hypervariable region is a portion of an antibody that physically binds to the polypeptide target. The antibody molecule is a glycoprotein comprising at least two light polypeptide chains and two heavy polypeptide chains, wherein each light and heavy chain contains a variable region located at the amino terminal portion of a polypeptide chain featuring an antigen-interaction region wherein the antigen is bound. The heavy and light polypeptide chains are also comprised of a constant region at the carboxy terminal portion.

The terms “genetic immunization” mean the injection or the delivery of a polynucleotide in a form and composition that is operatively expressible in an animal tissue to yield an immune response in a non-human animal. The expressed polynucleotide material will produce at least one transcript and possibly more than one transcript of different size depending on the start and stop sites in the polynucleotide sequence. Transcripts produced from the polynucleotide sequence will be translated into corresponding gene product(s) against which specific antibodies are made by the immune system of the animal in a sufficient quantity to be detectable and useable in biological assays.

“Monoclonal antibodies” are substantially homogenous populations of antibodies to a particular antigen obtained by any technique that leads to the production of antibody molecules by continuous cell lines in culture. Monoclonal antibodies may be obtained by methods known to those skilled in the art. See, for example, Kohler, et al., Nature 256:495-497 (1975), and U.S. Pat. No. 4,376,110.

The term “polyclonal” refers to antibodies that are heterogeneous populations of antibody molecules derived from the sera of animals immunized with an antigen or an antigenic functional derivative thereof. For the production of polyclonal antibodies, various host animals may be immunized by injection with the antigen. Various adjuvants may be used to increase the immunological response, depending on the host species.

The term “polynucleotide templates” means a mixture of polynucleotide sequences including RNA and/or DNA of different sizes and conformations, produced by different cell types of eukaryotic and prokaryotic origin. These sequences may be expressible in prokaryotic systems or in mammalian systems and may include their components as described herein.

The term “recombinant vector construct” means a composition of plasmid origin, viral origin, or a combination of both. Recombinant vector constructs may contain regulatory elements such as enhancers, promoters, kozak sequences, polyadenylation signals and the like, and allow operative expression of a polynucleotide sequence in mammalian cell type or tissue. Recombinant vector constructs may possess a sequence for the origin of replication in prokaryotic or eukaryotic systems and a gene that encodes for an antibiotic selection to confer resistance for the transformed organism with the recombinant vector construct. Recombinant vector constructs may contain sequences that facilitate the replication of the construct and/or facilitate the integration upon its delivery into a cell and/or a tissue of an animal, insect or a plant.

By “an array of antibodies” it is meant a group of antibodies bound to a solid support, where at least two of the antibodies in the array are directed to different binding partners. The antibodies are preferably arranged to form a line. However, the antibodies may also be arranged in any other formation, such as in a circle, a semi-circle, or to form shapes, such as X, [symbol] , or +, or any other shape.

By “specific binding affinity” is meant that the antibody binds to target polypeptides with greater affinity than it binds to other polypeptides under specified conditions. Antibodies having specific binding affinity to a gene product may be used in methods for detecting the presence and/or amount of the gene product in a sample by contacting the sample with the antibody under conditions to form an immunocomplex and to detect the presence and/or amount of the antibody conjugated to the gene product.

The cells, tissue samples, biological fluids or derivatives or extracts of these that are used in the invention are from an organism, either a single cell organism, such as bacteria, viruses, amoebae, or protozoa, or multicellular organisms, such as members of the plant and animal kingdoms. The organism is preferably a plant, an insect, or an animal. The plant is preferably selected from the group consisting of crops, such as grains, nuts, vegetables, and fruits, household plants, trees, and bushes. In most diagnostic applications, the sample is from a human patient and may include tissue from a normal or disease state such as a tumor, a biological fluid such as ascites, urine, plasma, serum, spinal or cerebral fluid, or other preparation and may be processed for advantageous use in the kits of the invention.

The method of the invention relates to expression of a polynucloetide sequence from any genome to generate polypeptide(s) against which the non-human animal makes specific antibodies. The method of the invention also includes the use of the produced antibodies in vitro and in vivo to gain valuable information on the function of the antibody, the antibody target, and the corresponding polynucleotide. The present invention includes any combination of the following steps of:

a) using a recombinant vector construct containing a polynucleotide or polynucleotide template;

b) delivering a polynucleotide template to an animal to generate antibodies to the expressable polypeptide made by the recombinant vector construct;

c) using the antibodies in multiplex formats such as protein arrays, antibody arrays, tissue arrays and the like, to analyze biological samples;

d) selecting antibodies against known and unknown gene products that correlate to a disease onset, a biological process or any other relevant biological information;

e) creating an array of the antibodies on a solid support;

f) contacting a sample from the organism to the array of antibodies so that the gene products bind to their respective antibodies in the array, where the binding can be measured;

g) determining the presence or absence, and if present, the amount of, the gene products bound to said antibodies;

h) correlating the results to the presence or absence of disease or to a relevant biological function; and

i) correlating the result of the expression of at least a polynucleotide sequence to the antibody, its target and the derived information.

In preferred embodiments, the organism is a human patient and the antibodies targets relate to a certain condition. The certain condition is preferably selected from the group consisting of suspicion of or screening for disease, progress of disease, or disease resistance, or response or resistance to treatment. The gene products may be obtained from the organism from any source tissue or sample as described herein.

In another specific aspect of the above invention, a method of diagnosing a disease in an organism is comprised the steps of: (a) producing antibodies against polypeptide of a mammal or a pathogen; (b) using the antibodies in a biological assay by contacting cell lysates from the organism to be analyzed using array of antibodies against the targeted mammal or pathogen, so that the gene products bind to their respective antibodies in the array, where the binding can be measured compared to a standard.

### EXAMPLE 1

### DNA Preparation Protocols—Existing Techniques and the Present Invention

Laboratory protocols and techniques to produce and prepare DNA or RNA materials from any organism, mammal, pathogen plant or others are well known to those of skill in the art. There are several alternative methods to prepare and purify DNA or RNA materials in Current Protocol in Molecular Biology, Volume 1, edited by Frederick M. Ausubel, 1996. In most conventional techniques, polynucleotide materials, DNA and RNA, are required to be highly pure to correlate the data to the effect of the polynucleotide materials and to avoid contaminating material in the polynucleotide solution that could interfere with the immunization. In these techniques, the process of polynucleotide purification requires several steps. Current laboratory methods to prepare plasmid DNA templates require extensive investments of time and resources. There are two widely used laboratory procedures for the preparation of lysate solution containing plasmid DNA: the boiling method and the alkaline lysis method. Both methods utilize laboratory scale centrifugation to separate cellular debris from the crude lysate. Organic extraction with phenol/chloroform/isoamyl alcohol or a variation of this mixture is typically used to improve the purification of the plasmid DNA template. Further purification of plasmid DNA template is improved by laboratory scale ultracentrifugation using cesium chloride and ethidium bromide for generally more than 15 hours, followed by several butanol extractions and dialysis for 48 hours. Because this procedure is labor intensive, it cannot be used to purify a large number of plasmid DNA template at the same time for use to create recombinant vector constructs.

In variations of the methodology described above, the crude lysate is treated with pancreatic RNAse followed by alkaline detergent treatment to reduce the presence of bacterial RNA. An organic extraction with phenol/chloroform is followed by ethanol precipitation, re-suspension and a second ethanol precipitation. This procedure of template DNA purification remains a time consuming process that limit its utilization to produce a large number of DNA templates. Alternatively, alkaline solution lysate of a crude cell extract is centrifuged to remove cell debris and the remaining solution is treated to precipitate the polynucleotide templates. The polynucleotide templates is resuspended in a Tris-HCl/EDTA buffer and passed through an exchange column for further purification. Again, this method remains a laboratory scale and time consuming and limited in scope.

Thus, prior art processes for genetic immunization use highly pure DNA templates produced through a series of technical manipulations.

In a preferred embodiment of the present invention, the process and the method described herein is rapid, economical, scalable and suitable to purify large numbers of polynucleotide templates for genetic immunization. A polynucleotide template is prepared with an expressible polynucleotide coding for an immunogenic translation product that is introduced into a vertebrate wherein the translation product is formed to elicit an immune response against the gene product. The polynucleotide template used for immunization pursuant to this invention is preferably DNA and/or RNA sequences, although a replicating or a non-replicating recombinant vector construct or an integrating sequence may be used.

The preparation of nucleotide template of different fragment of the same gene sequence may be used to perform genetic immunization with the aim of making monoclonal antibodies. Typically, a gene sequence encoding for a gene product is divided into small fragments of 20, 30, 40, 50, 60, 70, 80, 90 and 100 base pairs or any integral value in any of these ranges or any interval of 10 or 100, preferably between 20 and 1000, but possible between 1000 and 5000, or more. These fragments may or may not overlap between themselves. The polynucleotide template may be introduced into tissues of the body using an injectable solution. The preparation may further advantageously comprise a source of a cytokine which is incorporated into liposomes in the form of a polypeptide or as a polynucleotide translatable into a gene product. The polynucleotide immunization selectively elicits a humoral immune response, a cellular immune response, or a mixture of these.

Potentially, recombinant vector constructs are derived from plasmid, or RNA or DNA viral origin or a combination of both. Recombinant vector constructs may include prokaryotic and eukaryotic replication sequences, incorporate various origins of replication for both eukaryotic and prokaryotic systems. These vectors can also encompass a number of genetic elements to facilitate cloning and expression of selectable genes and/or polylinkers, promoters enhancers, leader peptide sequences and alike. The selection of vectors, origins, and genetic elements may vary according to need and the host cell, and is within the skill of workers in this art. A host cell can be among the following cells but not limited to them: bacteria, yeast, fungi, insect and mammalian cells, and plant cells. Preferred cells are microbial cells particularly E. coli. Any suitable strain of E. coli is contemplated in this invention. Likewise genes encoding diverse proteins or peptides, polypeptides, glycoproteins, phosphoproteins, etc, are also contemplated in the present invention. The inserted polynucleotide sequence encoding a polypepetide into the recombinant vector constructs may correspond to partial or complete cDNA, to genomic DNA fragment, to synthetic DNA, to polynucleotide sequences from any of the following genomes: mammalian genomes, pathogen genomes, plant and insect genomes, but not limited to these referred to genomes. These sequences may be obtained by using chemical synthesis or gene manipulation techniques.

The polynucleotide template for genetic immunization contains an operatively coding recombinant vector construct for a partial or a complete gene product. The polynucleotide operatively encodes for a gene product and has all the genetic information necessary for expression, such as a suitable promoter, enhancer, polyadenylation signal and the like. The polynucleotides may be partial or complete sequences of any gene. These polynucleotides can be administered by any method that delivers injectable or inhalation spray materials to the functional components of the immune system of an animal, such as by injection into the interstitial space of tissues such as muscle or skin, introduction into the circulation or into body cavities or by inhalation or insufflation, or injection into any tissue of the mammal that is exposed to the immune response. Preferably, the injection is inguinal. The pH of the preparation of the delivered material is suitably adjusted to physiological pH ranges.

The polynucleotide templates may or may not replicate into the recipient animal and may or may not integrate in the recipient cell genome and the template may contain integration-facilitating constructs, combined with lipids, viral particles, or other such compounds. These polynucleotide templates may be non-replicating DNA sequences, or may have been genetically engineered to possess specific replicating elements to insure the maintenance and replication of the desired delivered polynucleotide templates. These features will allow the production of at least one polypeptide material for extended periods. The polynucleotide sequences into the recombinant construct to be administered to the immune system of the animal may be prepared from eukaryotic polynucleotide sequence referred to as cDNA, or derived from genomic DNA of any organism (i.e. plant, pathogen, animal). Alternatively, a coding sequence can be generated following the amplification of a given coding polynucleotide sequence using PCR technology, and included in an appropriate recombinant vector construct. The polynucleotide sequence is preferably driven by strong eukaryotic promoter such as RSV, LTR, CMV, ACTIN, PGK and other regulatory element to achieve the highest possible expression of the administered polynucleotide sequence in eukaryotic cells and or an animal tissues.

Transformed prokaryotic cells with recombinant vector construct containing polynucleotide sequence may be plated on solid agar LB medium or any other appropriate medium. Such medium contains preferably an antibiotic to select only transformed cells with recombinant vector construct containing polynucleotide sequences. Preferably transformed cells with the recombinant vector construct containing polynucleotide sequences are bacteria. Resistant clones are picked up individually and transferred to 96 well plates containing the LB medium with the same antibiotic for selective growth. 96 well plates containing transformed bacterial clones are grown overnight and duplicated in two 96 well plates. The first 96 well plate receives 100 microliters of 80% glycerol in each well and is stored a −80° C. for further usage. Each clone from the second 96 well plate is duplicated into another 96 well plate of LB medium and grown overnight to prepare polynucleotide templates. Other multiwell plates or individual tubes may be used to grow up the transformed clones for polynucleotide template preparation. Grown transformed bacterial clones are centrifuged for 15 minutes at 3000 rpm. A bacterial pellet of each clone is lyzed and the precipitate of the cellular proteins and chromosomal DNA is harvested and removed. Alternatively, isopropanol (0.7 of the total volume of the lysate solution) is added to precipitate the polynucleotide material by centrifugation. The supernatant is discarded and polynucleotide template recovered from the plastic using 300 microliters of Tris-HCl 10 mM, EDTA 1 mM. Alternatively, the isopropanol step may be omitted.

In another embodiment of the present invention, transformed bacterial colonies with recombinant vector construct containing the polynucleotide sequence are grown, harvested by centrifugation and lyzed by a chemical or physical agent to disrupt the bacteria. Then the whole solution is delivered to an animal.

Alternatively, the polynucleotide sequence may be expressed in prokaryotic system, insect system or plant cells. In these cases, the recombinant construct containing the polynucleotide sequence is engineered to contain appropriate promoters and regulatory elements to achieve the highest expression of the contemplated polypeptide in the plant cells, insect cells or prokaryotic systems respectively. Such expression system for individual polynucleotide sequence may be harvested, lyzed with chemical solution or physical agent and delivered to an animal to generate antibodies to the desired expressed polypeptide.

### EXAMPLE 2

### Gentic (DNA or RNA) Immunization—Existing Techniques and Modifications of the Present Invention

DNA material has been used to induce a protective immune response in a mammal by injecting a DNA sequence in a non-integrating composition. See U.S. Pat. No. 5,589,466. In addition antibodies were generated through lipid mediated DNA delivery. See U.S. Pat. No. 5,703,055.

Traditionally, known genes are used with a full known coding sequence, a known starting codon, a known transcript, a known open reading frame and a known protein. The expression vector is designed to introduce the polynucleotide sequence exactly in the correct open reading frame and the known coding sequence is verified in vitro as being capable of making the expected transcript and protein. Literature emphasizes the need to optimize the expression of the expected protein in vitro using eukaryotic cells prior to using the recombinant expression construct for DNA-based immunization many such efforts fail to obtain the expression of the desired protein even after careful design of the desired expression vector.

The process of verifying the correct open reading frame is costly, time consuming and labor intensive and requires several experimental procedures. This process can be utilized only in cases when the scientist knows the full nucleotide sequence from start to finish. Therefore, to generate antibodies to large number of proteins of any genome, the following must be known: (a) all the genes of the desired proteins; (b) the full length of all the transcripts; (c) the potential open reading frame of each transcript; (d); the start and the stop codons of each transcript; (e) and the size of the expected gene product encoded by each transcript(s) to introduce the polynucleotide sequence in the correct context of an expression construct for DNA-based immunization. Furthermore, the investigator must optimize the expression of the desired protein in vitro and analyze the protein using molecular assays to identify the polypeptide.

Unfortunately, the accumulated sequences from the human genome and other organisms available in private and public databases do not contain the information referred to above which are required when using DNA immunization technology on a large scale. Consequently, current technologies and information make it impossible to generate antibodies to several thousand proteins of a given genome.

In contrast to other methods known in the prior art of DNA immunization, the present invention relates to the introduction of a polynucleotide sequence into a recombinant vector construct without necessarily knowing the full length sequence of the gene from which the polynucleotide sequence was derived. The recombinant vector construct contains all the regulatory elements necessary for the expression of a polynucleotide sequence either in prokaryotic or in eukaryotic cell. Therefore, the delivery of the recombinant construct containing the appropriate regulatory element in to favor the expression of the polynucleotide in eukaryotic or prokaryotic cell system will lead into the replication, transcription, translation and modification of the resulting transcript(s) and polypeptide(s), by the cellular proteins and enzyme machinery. When the polynucleotide sequence in the recombinant construct contains a start codon or even more than one, it will be transcribed and translated into polypeptides. Thus, when the recombinant construct containing the polynucleotide sequence is delivered to an animal capable of mounting a humoral response, the animal makes antibodies to the polypeptide expressed by the delivered recombinant construct. The present invention takes advantage of the in vivo cellular machinery of the animal and allows the high throughput production of antibodies to large number of different polynucleotide sequences.

Pursuant to this invention, non pharmaceutical-grade polynucleotide templates containing DNA or RNA or both from an organism of choice are formulated in a solution that is physiologically acceptable for genetic immunization of a mammal, but specially designed and formulated to produce a strong immunological reaction against the gene product encoded by the polynucleotide sequence. This process produces a recombinant vector construct composed of supercoiled, concatemeric, and relaxed DNA. The limited purification procedure is free of solvent, organic, or mutagenic solutions and involves limited manipulation. The method of the invention can be scalable and with the performance of limited DNA prep, can prepare hundreds and even thousands of different polynucleotide templates simultaneously using limited and disposable materials. In practical application, several thousand polynucleotide templates containing recombinant vector construct containing a polynucleotide sequence can be prepared every day based on this invention. Finally, the present invention is substantially more economical than current methods, and is suited to economic preparation of a large number of polynucleotide templates for genetic immunization.

The preferred methodology uses a high-throughput preparation of polynucleotide templates that contain a recombinant vector construct that harbors a partial or complete gene sequence of any gene derived from a plant, insect, pathogen, mammal, vertebrate, invertebrate or any other organism. The delivery of the polynucleotide materials, prepared by the present methodology, yields a high efficiency immune response yielding specific polyclonal antibodies against one or more, but preferably one, gene product encoded by the corresponding gene sequence. Alternatively, the polynucleotide template may be concentrated by adding isopropanol, followed by centrifugation. The gene product may be modified or administered in an adjuvant in order to increase its antigenicity. Methods of increasing the antigenicity of a gene product are well known in the art.

In a preferred embodiment of the invention, each animal receives a single injection of the polynucleotide template solution. A few weeks (5-8 weeks) after the first immunization, animals are bled and sera are harvested. Alternatively, spleens are removed from the immunized animal, preferably mice, for monoclonal antibody generation. Splenocytes are separated from the rest of cells and connective tissues and fused with mycloma cell lines such as Sp2/0. Hybrid clones are grown in an appropriate selection medium for 10 to 14 days and their medium supernatant is tested for the production of specific monoclonal antibodies against the polypeptide target. In general, techniques for preparing monoclonal antibodies and hybridomas are well known in the art (Campbell, “Monoclonal Antibody Technology: Laboratory Techniques in Biochemistry and Molecular Biology,” Elsevier Science Publishers, Amsterdam, The Netherlands, 1984; St. Groth et al., J. Immunol. Methods 35:1-21, 1980). The antibodies of the invention can then be used in biological assays, diagnostic and a like. For example, if the polynucleotide contained in the recombinant vector constructs were derived from human polynucleotide material the generated antibodies will be used on human protein, cell, sera or tissues. Alternatively, the polynucleotide material in the recombinant vector construct could be a cDNA material from liver, lung, brain or other tissues. In these cases, the generated antibodies will be primarily tested on the protein extract, cells or tissues of the liver or lung or brain accordingly. The antibodies of the present invention include monoclonal and polyclonal antibodies, as well as fragments of these antibodies, and humanized forms. Sera containing the antibodies is isolated from the immunized animal and is screened for the presence of antibodies with the desired specificity for a given gene product. Specific antibodies to given gene product in the assay can be detected using an anti-animal antibodies. Procedures for accomplishing such detection labeling are well-known in the art.

### EXAMPLE 3

### The Invention Enhances the Information Derived from the Antibody-Antigen Interaction

As noted above, an important feature of the present invention is that the antibodies produced by the claimed methods are directly linked on a one-to-one basis with a polynucleotide sequence encoding a polypeptide to which the antibody is specific. An important embodiment of the current invention is that the polynucleotide sequence in the recombinant vector construct may encode a polypeptide with a similar domain to another protein. In this case, when tested on a biological sample, the generated antibodies will react against its specific target, if it is present in the biological sample. By observing the reaction between the antibodies and their targets in a sample, the antibodies provided by this invention can be used to explore the function of known or unknown proteins and related compounds.

For example, the expression of a gene encoding an unknown protein may be studied in normal versus cancer clinical samples, using the antibodies generated through the methods of this invention, in immunoassays. When antibodies are generated to a larger number of gene products, the antibodies can be rationally organized and tested against samples from known sources to characterize differential expression of a multitude of individual proteins in, for example, normal versus diseased samples. The reaction of the proteins with the individual members of a rationally organized antibody array also leads to identification of a large number of gene products that are differentially expressed in the normal versus diseased state. Similarly, differential comparative analysis of gene expression in various cell types, tissue types, and developmental stages can be obtained pursuant the application of the antibodies to two physiological states differentiated by virtually any state or stimulus.

When a polyclonal sera with a particular reactivity of interest, such as reactivity to a cancer-specific protein is identified, monoclonal antibodies may be produced that display the reactivity of interest. Once monoclonal antibodies are obtained, they are used in the same manner as the polyclonals directly resulting from genetic immunization as described herein. In addition, the antibodies produced by the methods and systems of the present invention may be labeled for use in a diagnostic assay or antibody array to screen biological samples to discover disease specific targets. Labeling and detection systems for antibody reactivity are well known to those in the art of this field.

To construct an information-enhanced array, the antibodies prepared by the above-described techniques are immobilized in a rationally organized matrix on a solid support. Examples of such solid supports include, but are not limited to, plastics such as polycarbonate, complex carbohydrates such as agarose and sepharose, acrylic resins and such as polyacrylamide and latex beads. The solid support may also include glass, silica, silica gel, silicon wafer, silicone, plastics such as polyethylene, polystyrene, polyvinyl chloride (PVC), or polyvinyl pyrrolidone (PVP), nylon, TEFLON®, nitrocellulose, ceramic, fiber optic, and semiconductor materials. Techniques for coupling antibodies to such solid supports are well known in the art (Weir et al., “Handbook of Experimental Immunology” 4th Ed., Blackwell Scientific Publications, Oxford, England, Chapter 10, 1986; Jacoby et al., Meth. Enzym. 34, Academic Press, N.Y., 1974).

The antibody array comprises a group of antibodies, which group comprises at least two antibodies and preferably comprises a much larger number including values between 10 and 100 and integral values therein, values between 100 and 1,000, in integral values therein, as well as intervals of 10, values between 1,000 and 10,000, as well as integral values therein, and intervals of 10 or 100, and values between 10,000 to 100,000, and as high as an antibody to each expression product in the genome of any vertebrate, plant, or insect. Although each individual sample or aliquot of antibodies that forms the members of the individual array can contain an antibody of more than one specificity, it is preferred that each member or dot of the array contain an antibody of a single specificity. In preferred embodiments of the invention therefore, at least two of the antibodies are different and in preferred embodiments at least 10, or at least 100, or at least 1,000, or at least 10,000, or at least 100,000 of the antibodies have different specficities, and are reactive with different gene products. As above, all integral values and intervals between these ranges are expressly disclosed herein.

Furthermore, any numerical value in the above ranges corresponds to the number of antibodies that are coupled to a solid support to form the particular arrangement of the array. Preferably, duplicate quantities of the antibodies are arranged in a side-by-side fashion in the array to provide for reproducibility and a control. For purposes of diagnostics, the arrays may be organized on a physiological basis such that individual portions of the arrays reflect gene sequences having differential expression in different disease states. For example, an array may be constructed having gene expression products for a variety of diseases, wherein such expression products are known to be expressed in a biological fluid such as saliva, urine, serum, plasma, ascites, spinal or cerebral spinal fluid, etc. In such a rational organization, the location of binding events of antibody target(s) in a sample provides specific information about the physiological state of the underlying organism.

To prepare an array, working dilutions of source antibodies are made at 1:100 or more when needed, in tris buffered saline (TBS) containing 0.02% BSA, 0.02% sodium azide. Aliquots of the antibody solution are arrayed on wet nitrocellulose on 2 layers of blotting paper. Two layers of precut blotting paper are placed in Omni-tray and are soaked with TBS solution. A precut sheet of nitrocellulose is placed over the nitrocellulose. Excess wetting solution is drained by tilting it and excess liquid absorbed. The individual members of the arrays may be deposited manually or with a Robotic system (Genomic Solutions Flexys^{7m}) to construct the array. The arrayed blots are dried on blotting papers for 5 minutes at room temperature. The dry blots are placed in 0.3% hydrogen peroxide in TBS and rocked for 5 minutes on a vertical rotor. The blots are rinsed twice in TBS solution with rocking (2 rinses of 5 minutes each). The blots are treated with blocking buffer in blocking solution (2.5% Non Fat Dry Milk in TBST) in a wide tray for 1 hour with constant rocking at room temperature. After blocking, the blots are given 2 quick rinses with TBS solution for 2 minutes each.

Alternatively, other protocols using different labeling and detection systems can also be used within the scope of the present invention.

### EXAMPLE 4

### Gene Expression Profiling Using the Antibody Array

It is known in the art that in response to a condition or a disease, differential expression at specific genes of an organism occurs, giving rise to the presence of specific gene products in the organism's cells. For example, if an organism suffers from a viral or a bacterial infection, to combat or cope with the infection, the organism produces certain gene products. It is also known that the organism may produce the gene products specific to the condition before the organism itself shows any morphological signs of suffering. By way of example only, a person suffering from the common cold will produce specific gene products associated with the disease before the person notices a runny nose or watery eyes.

Similarly, in carcinomas, the up or down regulation of genes that cause or accompany the disease state will result in the differential expression of genes and the differential presence of gene products in a sample. The gene products contained in virtually any biological sample may be described as “cell contents” because such products are excreted, derived, or extracted from a cell source such as tissue, plasma, tumor cells, etc. To test whether an organism is suffering from that disease or condition, the cell contents are exposed to the antibody array. The binding events at the reaction sites of the antibody array enables the identification of the gene products excreted, derived, or extracted from the cell. These binding evens may be compared to known values for non-diseased patients or other controls indicating the absence of disease.

To perform the analysis, a test solution of biotinylated human serum (B-HS) and biotinylated blucose-6-phosphate dehydrogenase (B-G6PDH) is prepared in 10 ml volume per blot. For example, 100 ul B-Hs of 4 mg/ml stock concentration and 5 ul of 10 ug/ml stock solution of B-G6PDH are added in 10 ml of pre-mixed solution of 80:20 TBS blocking buffer, mixed thoroughly, and sonicated at high frequency for 20 minute at room temperature. The sonicated solution is added to the blot in an Omni-tray. The solution is first poured in the Omni tray (10 ml) and thoroughly spread in the Omni tray, and then the blocked and washed nitro-cellulose blots are placed therein. Each blot is incubated individually with the test solution for 1 hour at room temperature on the rocker. After incubating, the gene product sample solution is poured onto the array and rinsed with 10 ml of PBST (30 seconds each). Each blot is transferred to a Plexi-glass wash vessel having 30 ml of was solution (PBST). The blots are rotated on a Gyro-shaker (Speed—˜100 RPM) and washed 6 times with wash solution. For conjugate binding, a 1:10000 dilution of Neut-avidin-HRO conjugate in PBS is prepared with 10 ml of conjugate solution for each blot. The 10 ml of conjugate is dispensed in the Omni tray and rocked for 45 minutes or a vertical rocker. To analyze the initial or “primary” binding of gene products to the array, a positive IgG blot is washed twice with wash solution and incubated with 10 ml of 1:100,000 dilution of goat anti-mouse-IgG HRP in the Omni-tray while on the vertical rocker. After 45 minutes, the test blots and positive IgG blots are rinsed twice with PBST (2×30 seconds). The blots are placed in Plexibox wash vessel containing 30 ml of wash solution (PBST) and washed 6 times with wash solution. A chemiluminescent substrate is prepared by mixing equal volumes of the two solutions in a 50 ml polypropylene tube. About 7 ml of substrate solution are prepared for each immunoblot. The solutions are mixed in a dark room to avoid the light. Seven ml of substrate solution are dispensed in an Omni-tray and the immunoblot is placed in the tray, followed by incubation in the dark by rocking for 5 minutes on a Vertical rocker. After 5 minutes of incubation, the blots are placed in a plastic sheet cover and the sheet is placed in an x-ray cartridge. The film for various progressive times to gauge the appropriate exposure time (from few minutes to 30 minutes) to achieve dark spots without complete saturation of the spots.

### EXAMPLE 5

### Disease Specific Antibody Arrays

One important embodiment of the present invention is to use the antibodies generated by this invention in multiplex screening analyses. Antibodies can also be divided based on their specificity to form antibody arrays for cell types, tissue types, disease types, for toxicology, pharmacology, for chemiopharmacology, exposure to any physical or chemical agent, etc. Specific antibodies arrays are used to perform comparative analysis of the desired biological samples.

In each case, practice of the invention enables one to identify the differential expression of proteins that comprise both the causative agents of the underlying disease state, as well as physiological events that are components of the overall biological cascade resulting from any disease state or exposure to stimulus. Once the differential expression profiles are obtained, the gene sequences and corresponding gene products can be identified and further studied for use as markers, as well as diagnostic or therapeutic products.

### EXAMPLE 6

### Identification of Antibodies Targets of Specific Pathways

To analyze pathway differential gene expression in response to external stimulus, normal and diseased biological samples are analyzed by exposing the proteins of the sample to the antibody arrays. The selected antibodies used to construct the array may or may not be specially selected for the indication.

For example, using the same protocol, the gene expression of varying cell types can be analyzed following exposure to hormones, growth factors, bioactive chemicals generally, drugs, especially chemotherapy compounds, and virtually any toxin or agent whose effect on cell growth or metabolism and the underlying gene expression is of interest.

It will be readily apparent to one skilled in the art that varying substitutions and modifications may be made to the invention disclosed herein without departing from the scope and spirit of the invention. All patents and publications are herein incorporated by reference to the same extent as if each individual publication was specifically and individually indicated to be incorporated by reference.

I claim:

1. A method of producing antibodies in a non-human vertebrate, the method comprising injecting the vertebrate with a composition comprising:
an extracellular deoxyribonucleic acid sequence comprised of genomic DNA, bacterial ribonucleic acid,
and a pharmaceutically acceptable carrier; expressing in the non-human vertebrate sequence encoded by the genomic DNA; collecting isotype IgG antibodies from the non-human vertebrate.

2. The method of claim 1, wherein the antibodies are harvested within seven weeks of the first injection of the composition into the vertebrate.

3. The method of claim 2, wherein the antibodies are harvested within six weeks of the first injection of the composition into the vertebrate.

4. The method of claim 1, wherein the vertebrate is injected a single time with the composition.

5. The method of claim 1, wherein the vertebrate is injected inguinally.

6. The method of claim 1, wherein the vertebrate is a mammal.

7. The method of claim 6, wherein the mammal is an outbred mouse.

8. The method of claim 1, wherein the weight ratio of the bacterial ribonucleic acid to deoxyribonucleic acid in the composition is at about 1/10,000 to about 1/1.

9. The method of claim 8, wherein the weight ratio of the bacterial ribonucleic acid to deoxyribonucleic acid in the composition is about 1/1000 to about 1/1.

10. The method of claim 9, wherein the weight ratio of the bacterial ribonucleic acid to deoxyribonucleic acid in the composition is about 1/100 to about 1/1.

11. The method of claim 1, wherein the composition comprises about 50 μg to about 500 μg deoxyribonucleic acid.

12. The method of claim 11, wherein the composition comprises about 200 μg to about 450 μg deoxyribonucleic acid.

13. The method of claim 12, wherein the composition comprises about 250 μg to about 400 μg deoxyribonucleic acid.
